(19) United States
(12) Patent Application Publication
(10) Pub. No.: US 2023/0310446 A1
(43) Pub. Date: Oct. 5, 2023

(54) RHO KINASE INHIBITION FOR TREATMENT OF PROLIFERATIVE VITREORETINOPATHY AND CONDITIONS ASSOCIATED WITH EPITHELIAL TO MESENCHYMAL TRANSITION

(71) Applicant: The Schepens Eye Research Institute, Inc., Boston, MA (US)

(72) Inventors: Leo A. Kim, Brookline, MA (US); Joseph F. Arboleda-Velasquez, Newton, MA (US); Dhanesh Amarnani, Allston, MA (US)

(21) Appl. No.: 18/024,623

(22) PCT Filed: Sep. 7, 2021
(86) PCT No.: PCT/US2021/049322

§ 371 Date: Mar. 3, 2023

Related U.S. Application Data
(60) Provisional application No. 63/175,383, filed on Apr. 15, 2021.
(60) Provisional application No. 63/146,092, filed on Feb. 5, 2021.
(60) Provisional application No. 63/078,387, filed on Sep. 15, 2020.
(60) Provisional application No. 63/075,157, filed on Sep. 6, 2020.

(51) Int. Cl. A61K 31/551; A61K 9/00; A61K 47/38; A61K 31/472; A61P 27/02

(52) U.S. Cl. CPC A61K 31/551; A61K 9/0048; A61K 47/38; A61K 31/472; A61P 27/02

(57) ABSTRACT

The use of Rho Kinase (ROCK1/2) inhibitors for treating or reducing risk of proliferative vitreoretinopathy (PVR) or epiretinal membranes (ERM), e.g., after surgical vitrectomy to treat retinal detachment, and for treatment or reducing risk of conditions associated with epithelial to mesenchymal transition (EMT), including ocular fibrosis.

### CLAIM OF PRIORITY

This application claims the benefit of U.S. Provisional Patent Application Serial Nos. 63/075,157, filed on Sep. 6, 2020; 63/078,387, filed on Sep. 15, 2020; 63/146,092, filed on Feb. 5, 2021; and 63/175,383, filed on Apr. 15, 2021. The entire contents of the foregoing are hereby incorporated by reference.

### TECHNICAL FIELD

This invention relates to the use of Rho Kinase (ROCK1/2) inhibitors for treating or reducing risk of proliferative vitreoretinopathy (PVR) or epiretinal membranes (ERM), e.g., after open globe injury, surgical vitrectomy and/or scleral buckle to treat retinal detachment, and for treatment or reducing risk of conditions associated with epithelial to mesenchymal transition (EMT), including ocular fibrosis.

### BACKGROUND

Retinal detachment (RD) is an important cause of sudden visual loss in the United States, with approximately 40,000 cases occurring annually. Permanent visual loss will result if treatment is delayed.

A retinal detachment is defined as the separation of the neurosensory retina from the retinal pigment epithelium (RPE). In the nonpathologic state, the retinal pigment epithelium is a continuous epithelial monolayer occluded by tight junctions, which maintain a strict separation of the underlying choroidal capillary beds from the photoreceptors of the sensory retina, thus forming the outer blood-retina barrier. Its functions include the nourishment of photoreceptors, elimination of waste products, and reabsorption of subretinal fluid.

The definitive treatment of retinal detachment is surgical repair. Multiple operative techniques are available to the treating retinologist, but the principles underlying treatment of retinal detachment remain the same: removal of fluid from the subretinal space, relief of any existing traction, and treatment and prophylaxis against the underlying cause for the ingression of fluid, whether it be due to a retinal break or an exudative process.

Proliferative vitreoretinopathy (PVR) is the most common cause for failure of retinal detachment surgery, a complication which occurs in 5-10% of all retinal detachment surgeries. PVR can also occur spontaneously in the absence of surgery. PVR is most likely to develop following repeated surgical instrumentation of the eye, following significant physiologic insult to the eye such as in trauma, as well as in retinal detachments complicated by multiple tears, giant tears, vitreous hemorrhage, or in eyes with uveitis. PVR is a “scarring” condition that forms inside the eye after surgery, significant trauma, or even spontaneously. Its pathogenesis is the disruption of the retinal pigment epithelium layer, which is associated with inflammation, migration, and proliferation of cells to the (neural) retinal surface. Over the next 4-12 weeks, membranes on the surface of the retina proliferate, contract, and apply traction on the retina, which results in redetachment of the retina from the RPE. Once PVR is present and the retina detaches for a second time, it is unlikely that vision will be restored. PVR is most likely to develop following repeated surgical procedures of the eye, following significant physiologic insult to the eye such as in trauma, as well as in retinal detachments complicated by multiple tears, giant tears, vitreous hemorrhage, or in eyes with uveitis. PVR is especially prevalent after retinal detachment associated with open globe injury, where it occurs in approximately 50% of cases (Colyer et al. Ophthalmology. 2008; 115:2087-2093; and Eliott et al., Retina. 2017; 37:1229-1235). PVR is also a common complication of post-traumatic eye surgery. In this case, cells also grow uncontrollably beneath or on top of the retina triggering pre/sub-retinal membrane formation, tractional retinal detachment, and permanent vision loss. PVR occurs in 40-60% of patients with open globe injury. Hence PVR is highly relevant for the military and military-related eye trauma (Colyer et al., Ophthalmology, 2008.115(11): p. 2087-93).

A milder form of PVR, called macular pucker or epiretinal membrane (ERM), complicates the post-operative course of 20-30% of RD surgeries and half of these are so visually distorting that patients will require surgery. Epiretinal membranes (ERM) are caused by an abnormal proliferation of cells, e.g., retinal pigment epithelial (RPE) cells, glial cells, fibroblasts, and macrophages, on the surface of the retina, typically in response to ocular disease; the membranes tend to contract and cause puckering and thus distortion of the macula. See, e.g., Hiscott et al., Br J Ophthalmol. 68(10):708-15 (1984); Hiscott et al., Eye 16, 393-403 (2002); and Asato et al., PLoS One. 8(1): e54191 (2013).

### SUMMARY

Provided herein are methods for treating or reducing the risk of proliferative vitreoretinopathy (PVR) or epiretinal membranes (ERM), or a condition associated with epithelial to mesenchymal transition (EMT), in a subject, the method comprising administering a therapeutically effective dose of a ROCK1/2 inhibitor. Also provided are compositions comprising a ROCK1/2 inhibitor, for use in methods of treating or reducing the risk of proliferative vitreoretinopathy (PVR) or epiretinal membranes (ERM), or a condition associated with epithelial to mesenchymal transition (EMT), in a subject.

In some embodiments, the ROCK1/2 inhibitor is formulated to be administered by intravitreal injection. In some embodiments, the methods include administering an intravitreal injection of a ROCK1/2 inhibitor.

In some embodiments, the ROCK1/2 inhibitor is administered posterior to the limbus.

In some embodiments, the subject has age-related macular degeneration (AMD)/scarring occurring in association with macular degeneration or is undergoing an ocular surgical procedure that increases the subject's risk of developing ERM or PVR.

In some embodiments, the ocular surgical procedure is a pars plana vitrectomy (PPV), Retinal Detachment (RD) surgery; ERM surgery; scleral buckle surgery; or a procedure in the other eye.

In some embodiments, the condition associated with epithelial to mesenchymal transition (EMT) is a condition described herein, e.g., an ocular condition.

In some embodiments, the subject requires a PPV to treat a primary rhegmatogenous retinal detachment; rhegmatogenous retinal detachment secondary to trauma; preexisting proliferative vitreoretinopathy; or has other indications associated with high risk condition for PVR development.

In some embodiments, the indication associated with high risk condition for PVR development is a giant retinal tear, a retinal break larger than 3 disc areas, a long-standing retinal detachment, or a detachment associated with hemorrhage.

In some embodiments, a first injection is given at conclusion of the surgical procedure; and at least one, two, three, four, or more weekly injections are given postoperatively.

In some embodiments, the methods include intravitreally administering a sustained release formulation of ROCK1/2 inhibitor. In some embodiments, the sustained release formulation is or comprises a lipid-encapsulated formulation; multivesicular liposome (MVL) formulations; nano- or microparticles; polyion complex (PIC) micelles; or bioadhesive polymers. In some embodiments, the bioadhesive polymers comprise one or more of hydroxypropyl methylcellulose (HPMC), carboxymethylcellulose (CMC), polyacrylic acid (PAA), or hyaluronic acid (HA).

In some embodiments, the inhibitor reduces the extent of or reverses proliferative vitreoretinopathy (PVR) or epiretinal membranes (ERM).

Also provided herein are pharmaceutical compositions for ophthalmic uses comprised of a therapeutically effective amount of a Rho Kinase inhibitor in, or encapsulated with, a pharmaceutically acceptable carrier or vehicle. In some embodiments, the carrier or vehicle includes a sterile liquid medium, compatible with the eye, that effectively solubilizes said Rho Kinase inhibitor in physiological active form. In some embodiments, the carrier or vehicle includes a lipid-based system conveying said Rho Kinase Inhibitor wherein said vehicle is selected from, but not limited to, liposomes, micelles, exosomes, lipid emulsions or lipid-drug complexes. In some embodiments, the carrier or vehicle includes a particle/polymer based system or vehicle conveying said Rho Kinase Inhibitor wherein said vehicle is selected from, but not limited to, nanoparticles, microparticles, polymer microspheres, or polymer-drug conjugates.

In some embodiments, the Rho Kinase Inhibitor is one of the following: (a) Ripasudil; (b) Netarsudil; (c) Fasudil; and/or (d) Y27632 (or a combination thereof).

Also provided are methods for treating an ocular disease or pathology associated with an epithelial to mesenchymal transition in the back of the eye comprised of the administration to said eye of a therapeutically effective amount a Rho Kinase inhibitor.

In some embodiments, the administration to said eye is by means of (but not limited to) topical application, suprachoroidal injection, intravitreal injection, posterior implant or intra ocular injection. In some embodiments, the topical application is by eye drops. In some embodiments, the ocular disease or pathology includes, but is not limited to, epiretinal membrane, proliferative vitreoretinopathy, or age-related macular degeneration.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a set of images showing that Rho-Kinase inhibition in TGF-β2 treated C-PVR cells reduces mesenchymal phenotypic change.

FIGS. 2A-B show that rho-Kinase inhibitors decrease proliferation of C-PVR cells. (A) At 48 hours, ripasudil, netarsudil and fasudil significantly reduced proliferation in C-PVR cells by 44%, 95%, and 20% respectively at the highest concentration (10 μM), 25%, 37% and 39% percent reduction respectively with the lower concentration (1 μM), 21%, 39%, and 24% percent reduction respectively with the lowest concentration (0.1 μM). (B) Low levels of cell death were detected by LDH analysis at all concentrations of ripasudil treatments and at low concentrations of netarsudil treatments (0.1 μM).

FIG. 3 shows rho-kinase inhibitors ripasudil, netarsudil and fasudil significantly reduced migration in C-PVR cells by 65%, 100%, and 40% respectively at a 1 μM concentration for all drugs.

FIG. 4 shows that TGF-β2 treatment in C-PVR induces RhoA activation. TGF-β2 activation of RhoA had a 1.5 increase over control 15 minutes after stimulation, a 2 fold increase over control at 30 minutes, and a slight decrease over control at 60 minutes.

FIGS. 5A-B are images and a graph, respectively, showing that Rho-Kinase inhibitors decrease proliferation and migration of C-PVR cells. Robust outgrowths were observed growing from the freshly isolated PVR explant samples at 7 and 14 days (28.58 mm and 207 mm respectively) post embedding in Matrigel in culture. Ripasudil (0.8 mm and 15 mm) and netarsudil (4.2 mm and 37 mm) successfully inhibited and reduced explant growth at 7 and 14 days. The explants treated with fasudil (1 μM) and Y-2762 (1 μM) showed no outgrowths and almost complete inhibition of proliferation and migration at all time points.

FIG. 6 shows that treatment with ROCK inhibitors reduced TGF-beta-induced increases in N-cadherin and fibronectin (markers of mesenchymal transition).

### DETAILED DESCRIPTION

Described herein are methods for treating or reducing risk of PVR or ERM in a subject who is a retinal hole or a retinal tear, the method comprising administering to the subject a Rho Kinase (ROCK1/2) inhibitor. Without wishing to be bound by theory, the present methods reduce, or reduce risk of, proliferation or migration or Epithelial-mesenchymal transition (EMT) of retinal pigment epithelial (RPE) cells or other cells including retinal glial cells, macrophages, and fibroblasts, or EMT of retinal pigment epithelial cells, corneal epithelial cells, conjunctival epithelial cells, and other cells within the eye. The role of EMT in PVR and other conditions is discussed in US 20200377888. See also PCT/US2017/061620; PCT/US2018/061110; PCT/US2018/061156; and PCT/US2015/042951, all of which are incorporated herein by reference.

The present methods were developed in patient-derived in vitro models of PVR, created using cells from patients with PVR, which can be used to assess and screen for drugs as potential treatment of PVR. As shown herein, Rho kinase inhibitors (also referred to herein as ROCK or ROCK1/2 inhibitors) had a significant effect on the proliferation of PVR cells in vitro. Use of Rho kinase inhibitors significantly decreased cell proliferation of PVR cells, which is a hallmark of this condition. In addition, single cell RNA sequencing of 3 patient-derived PVR membranes revealed extensive expression of Rho kinase A and B within patient-derived PVR membranes.

Subjects

The methods described herein can be used to prevent (reduce the risk of) conditions associated with EMT, e.g., for reduction, treatment, or prevention of aberrant or pathological EMT occurring in the eye, in subjects having a condition as described herein. Suitable subjects can be identified using methods known in the art.

The methods can be used or to prevent (reduce the risk of) PVR or ERM in subjects, e.g., in subjects requiring pars plana vitrectomy (PPV), e.g., for subjects with spontaneous rhegmatogenous retinal detachment or rhegmatogenous retinal detachment secondary to trauma; for subjects requiring PPV for preexisting proliferative vitreoretinopathy grade C or higher; for subjects with retinal detachments requiring PPV for other indications associated with high risk condition for PVR development, e.g., giant retinal tears (giant retinal tears are defined as tears involving 90° or more of the circumference of the globe), retinal breaks larger than 3 disc areas, long-standing retinal detachments, or detachments associated with hemorrhage; in subjects who have suffered an open globe injury; in subjects who develop ERMs after an ocular surgery including cataract and glaucoma surgery; and/or in subjects with age-related macular degeneration (AMD)/scarring occurring in association with macular degeneration. PVR and ERM can be diagnosed by methods known in the art, e.g., the observation of cell outgrowths, membranes and bands in the vitreous during an ophthalmological exam, fundus or optical coherence tomography (OCT).

Other uses of ROCK1/2 inhibitors in the eye in addition to PVR include the following:

Prevention of Epiretinal Membranes after Retinal Detachment (RD) Surgery

Approximately 20-30% of RD cases develop clinically perceptible ERMs. Half of these are so visually distorting that patients will require surgery. In addition, autopsy studies show that close to 75-80% of patients with RD surgery have some degree of proliferation of membranes. This may explain why many patients do not achieve perfect vision postoperatively after RD surgery, yet do not have any ERMs grossly perceptible to the human eye.

Prevention of ERMs that Develop Spontaneously

ERMs can develop spontaneously, which then requires surgery. If a subject developed an ERM in one eye, a Rho kinase inhibitor may be used to prevent the development of ERMs in the other eye.

Prevention of Secondary ERM after ERM Surgery

For patients who develop ERMs, these can be removed but some reoccur and require reoperation. The present methods can be used to reduce the risk of recurrent ERM.

The methods described herein can include identifying and/or selecting a subject who is in need of treatment as described herein, e.g., to reduce the risk of development of PVR or ERM as a result of a condition listed above (e.g., selecting the subject on the basis of the need of treatment as a result of a condition listed above, e.g., an increased risk of developing PVR or ERM as a result of a condition listed above). In some embodiments, the subject does not have glaucoma, elevated Intraocular Pressure (TOP), corneal damage, cataracts, or diabetic retinopathy/proliferative diabetic retinopathy (PDR). See, e.g., Moshirfar et al., Med Hypothesis Discov Innov Ophthalmol. 2018 Fall; 7(3): 101-111.

The presentation of PVR clinically encompasses a wide phenotype. PVR can vary from a mild cellular haze (Grade A) to thick, fibrous membranes that cause the characteristic stiffened funnel of the detached retina (Grade D). A number of grading systems are in use, see, e.g., Ryan, Retina, 5^{th} ed (Elsevier 2013); Retina Society Terminology Committee, Ophthalmology 1983; 90:121-5 (1983); Machemer et al., Am J Ophthalmol 112:159-65 (1991); Lean et al. Ophthalmology 1989; 96:765-771. In some embodiments the methods include identifying, selecting, and/or treating a subject who has a low grade (e.g., Grade A or Grade 1) PVR, or who has ERM. In some embodiments, the methods include monitoring the subject for early signs of the development of PVR or ERM, i.e., the presence of a “vitreous haze” indicating a cellular proliferation (which may eventually develop into an organized sheet), and administering one or more doses of a ROCK1/2 inhibitor as described herein. Although early Grade A PVR vs. an early ERM may be difficult to distinguish from one another, eventually untreated PVR will progress; ERMs will cause a mild traction on the macula resulting in metamorphopsia but will not cause detachment of the retina, whereas untreated PVR will cause detachment and eventually result in a funneled, atrophic retina. The methods can also be used to treat subjects without present signs of PVR but who are at risk for PVR or ERMs.

Methods of Treating or Reducing Risk of PVR or ERM, or Conditions Associated with EMT

The methods described herein include the use of ROCK1/2 inhibitors in subjects who are at risk of developing a first or recurring PVR or ERM, e.g., a subject who is undergoing ocular surgery, e.g., RD surgery or ERM surgery, as described above, and in subjects who have PVR or ERM or who are at risk for developing PVR or ERMs. In some embodiments, the methods described herein include the use of ROCK1/2 inhibitors in subjects who have undergone, are undergoing, or will undergo a pars plana vitrectomy (PPV) or scleral buckle (SB). In some embodiments, the methods include performing a PPV, RD surgery, or ERM surgery. Methods for performing these surgeries are known in the art; for example, typically, PPV is performed under local or general anesthesia using three, 23 or 20 gauge sclerotomy ports. Any present epiretinal membranes can be dissected, e.g., using a membrane pick and forceps. Intraoperative tissue staining, perfluorocarbons, cryopexy, endolaser, scleral buckling, and lensectomy can also be performed as needed. Standard tamponading agents can be used, e.g., silicone oil or gas.

ROCK1/2 inhibition is useful for reduction, treatment, or prevention of aberrant or pathological EMT occurring in the eye. For example, the methods described herein are used for reducing proliferation and migration of cells within the eye undergoing epithelial to mesenchymal transition, e.g., inhibitors are administered to subjects diagnosed with, suffering from, or having EMT-associated diseases of pathologic ocular fibrosis and proliferation. Thus, the methods described herein include the use of ROCK1/2 inhibitors in subjects who have other conditions associated with EMT including cancer, e.g., mesothelioma; Ocular Chronic Graft-Versus-Host Disease, corneal scarring, corneal epithelial downgrowth, conjunctival scarring, eye tumors like melanoma, ocular fibrosis, fibrosis, and complication of glaucoma surgery and/or aberrant post-surgical fibrosis (e.g. after glaucoma surgery, cataract surgery, LASIK, or any intraocular surgery, e.g., post-surgical fibrosis as described in, e.g., Masoumpour et al., Open Ophthalmol J. 2016 Feb. 29; 10:68-85), fibrosis, glaucoma (Friedlander et al., J Clin Invest. 2007, Mar. 1; 117(3): 576-586); conjunctival fibrosis (e.g., ocular cicatricial pemphigoid), as well as orbital fibrosis as found in thyroid eye disease (Graves' disease).

The methods described herein include the use of an effective amount of a ROCK1/2 inhibitor. An “effective amount” is an amount sufficient to effect beneficial or desired results, e.g., the desired therapeutic effect (i.e., a prophylactically effective amount that reduces the risk of developing PVR or ERM). An effective amount can be administered in one or more administrations, applications or dosages. The compositions can be administered one from one or more times per day to one or more times per week to one or more times per month; including once every other day. The skilled artisan will appreciate that certain factors may influence the dosage and timing required to effectively treat a subject, including but not limited to the severity of the disease or disorder, previous treatments, the general health and/or age of the subject, and other diseases present.

In some embodiments, intravitreal ROCK1/2 inhibitor injections are performed aseptically after the topical application of anaesthesia and an antiseptic agent, e.g., 5% povidone iodine, to the conjunctival sac. In some embodiments, each subject receives an intravitreal injection of a ROCK1/2 inhibitor, e.g., 3.0 to 3.5 mm posterior to the limbus, depending on lens status, with a 30-gauge needle.

In some embodiments, the subjects receive one or more intravitreal injections of a ROCK1/2 inhibitor during their post-operative period. The first injection can be administered intraoperatively; subsequent injections can be administered, e.g., weekly or monthly.

In some embodiments, the subjects receive a sustained release implant, e.g., as described above, that will release a ROCK1/2 inhibitor over time, e.g., over a week, two weeks, a month, two months, three months, six months, or a year. In some embodiments, the methods include administering subsequent implants to provide administration of the ROCK1/2 inhibitor for at least six months, one year, two years, or more.

ROCK1/2 Inhibitors

A number of small molecule inhibitors of ROCK1/2 are known in the art and can be used in the present methods and compositions including cyclohexanecarboxamides such as Y-27632 ((+)-(R)-trans-4-(1-aminoethyl)-N-(4-pyridyl)cyclohexanecarboxamide dihydrochloride) and Y-30131 ((+)-(R)-trans-4-(1-aminoethyl)-N-(1H-pyrrolo[2, 3-b]pyridin-4-yl)cyclohexanecarboxamide dihydrochloride)(see Ishizaki et al., Mol Pharmacol. 2000 May; 57(5):976-83), as well as Y-30141, Y-33075, and Y-39983; dihydropyrimidinones and dihydropyrimidines, e.g., bicyclic dihydropyrimidine-carboxamides (such as those described in Sehon et al. J. Med. Chem., 2008, 51 (21): 6631-6634 and US2018/0170939); ureidobenzamides such as CAY10622 (3-[[[[[4-(aminocarbonyl) phenyl]amino]carbonyl]amino]methyl]-N-(1, 2, 3, 4-tetrahydro-7-isoquinolinyl)-benzamide); Thiazovivin; GSK429286A; RKI-1447 (1-(3-Hydroxybenzyl)-3-(4-(pyridin-4-yl)thiazol-2-yl)urea); GSK180736A (GSK180736); Hydroxyfasudil (HA-1100); OXA 06; Y-39983; Netarsudil (AR-13324, see Lin et al., J Ocul Pharmacol Ther. 2018 Mar. 1; 34(1-2): 40-51, U.S. Pat. Nos. 8,450,344 and 8,394,826); GSK269962/GSK269962A; Fasudil (HA-1077, 1-(5-isoquinolinesulfonyl)-homopiperazine) and its derivatives such Ripasudil (K-115, 4-fluoro-5-[[(2S)-2-methyl-1,4-diazepan-1-yl]sulfonyl]isoquinoline; see WO1999/20620) and others that share the core structure of 5-(1,4-diazepan-1-ylsulfonyl)isoquinoline; KD025 (SLx-2119) and related compound and XD-4000 (see, e.g. Liao et al. 2007 J Cardiovasc Pharmacol 50:17-24; WO2010/104851 US 2012/0202793); SR 3677; AS 1892802; H-1152 ((S)-(+)-2-Methyl-1-[(4-methyl-5-isoquinolinyl)sulfonyl] homopiperazine, Ikenoya et al., J. Neurochem. 81:9, 2002; Sasaki et al., Pharmacol. Ther. 93:225, 2002); N-(4-Pyridyl)-N′-(2,4,6-trichlorophenyl)urea (Takami et al., Bioorg. Med. Chem. 12:2115, 2004); and 3-(4-Pyridyl)-1H-indole (Yarrow et al., Chem. Biol. 12:385, 2005); 3-[2-(aminomethyl)-5-[(pyridin-4-yl)carbamoyl]phenyl] benzoates including AMA0076 (compound 32, Boland et al., Bioorganic & Medicinal Chemistry Letters 23(23): 6442-6446 (2013)); TCS-7001, BA-210, β-Elemene, Chroman 1, (5Z)-2-5-(1H-pyrrolo[2,3-b]pyridine-3-ylmethylene)-1,3-thiazol-4(5H)-one (DJ4), GSK-576371, GSK429286A, LX-7101, Verosudil (AR-12286), and AT13148, and pharmaceutically acceptable salts thereof. Inhibitors with the scaffold 4-Phenyl-1H-pyrrolo[2,3-b]pyridine, including compound TS-f22, are described in Shen et al., Scientific Reports 5:16749 (2015). Other ROCK1/2 inhibitors include Rhodblocks 1a-8 (described in Castoreno et al., Nat Chem Biol. 2010 June; 6(6): 457-463), isoquinoline sulfonyl derivatives disclosed in WO 97/23222, Nature 389, 990-994 (1997) and WO 99/64011; heterocyclic amino derivatives disclosed in WO 01/56988; indazole derivatives disclosed in WO 02/100833; pyridylthiazole urea and other ROCK1/2 inhibitors as described in 20170049760; and quinazoline derivatives disclosed in WO 02/076976 and WO 02/076977; in WO02053143, p. 7, lines 1-5, EP1163910 A1, p. 3-6, WO02076976 A2, p. 4-9, preferably the compounds described on p. 10-13 and p. 14 lines 1-3, WO02/076977A2, the compounds I-VI of p. 4-5, WO03/082808, p. 3-p. 10 (until line 14), the indazole derivatives described in U.S. Pat. No. 7,563,906 B2, WO2005074643A2, p. 4-5 and the specific compounds of p. 10-11, WO2008015001, pages 4-6, EP1256574, claims 1-3, EP1270570, claims 1-4, and EP 1 550 660. These inhibitors are generally commercially available, e.g., from Santa Cruz Biotechnology, Selleck Chemicals, and Tocris, among others. For example, fasudil and Hydroxy fasudil are obtainable from Asahi Kasei Pharma Corp (Asano et al., J Pharmcol Exp Ther, 1987, 241(3):1033-1040), Y-39983 is obtainable from Novartis/Senju (Fukiage et al., Biochem Biophys Res Commun, 2001, 288(2):296-300) and Y27632 is obtainable from Mitsubishi Pharma (Fu et al., FEBS Lett, 1998, 440(1-2):183-187). (S)-(+)-2-Methyl-1-[(4-methyl-5-isoquinolinyl) sulfonyl]homopiperazine], N-(4-Pyridyl)-N′-(2,4,6-trichlorophenyl) urea and 3-(4-Pyridyl)-1H-indole are also available at AXXORA (UK) Ltd and other suppliers. Protein or peptide inhibitor of ROCK1/2 are also known in the art, including inhibitors of ROCK1/2, e.g., a peptide consisting of 4-30 residues and exhibiting the sequence YSPS (SEQ ID NO:1), ERTYSPS (SEQ ID NO:2), or ERTYSPSTAVRS (SEQ ID NO:3)(see, e.g., US20170296617), or a kinase-defective mutant of ROCK1 or caspase 3 cleavage-resistant mutant of ROCK1 (e.g., as described in 2006/0142193). In some embodiments, the peptide further comprises one or more, e.g., all, D-amino acid residues.

In some embodiments, the ROCK1/2 inhibitor is formulated, e.g., in Balanced Salt Solution.

In some embodiments, the ROCK1/2 inhibitor is netarsudil, formulated as 0.2 mg of netarsudil (equivalent to 0.28 mg of netarsudil dimesylate), with Benzalkonium chloride, e.g., 0.015%, added as a preservative, and inactive ingredients, e.g., comprising boric acid, mannitol, sodium hydroxide to adjust pH, and water for injection.

In some embodiments, the ROCK1/2 inhibitor is an eye drop solution comprising 0.4% ripasudil, equivalent to 4 g of ripasudil per 1000 mL of solution, with a preservative (e.g., benzalkonium chloride), and inactive ingredients, e.g., sodium dihydrogen phosphate anhydrous, glycerine, and sodium hydroxide, pH: 5.0-7.0.

For the treatment of an ocular disorder, a Rho kinase inhibitor (e.g., a pharmaceutical composition comprising a Rho kinase inhibitor) may be administered locally, e.g., as a topical eye drop, peri-ocular injection (e.g., sub-tenon), intraocular injection, intravitreal injection, retrobulbar injection, intraretinal injection, subretinal injection, suprachoroidal, subconjunctival injection, or using iontophoresis, or peri-ocular devices which can actively or passively deliver drug.

Sustained release of drug may be achieved by the use of technologies such as devices, implants (e.g., solid implants) (which may or may not be bio-degradable) or bio-degradable polymeric matrices (e.g., micro-particles). These may be administered, e.g., peri-ocularly or intravitreally.

Pharmaceutical formulations adapted for topical administration may be formulated as aqueous solutions, ointments, creams, suspensions, lotions, powders, solutions, pastes, gels, sprays, aerosols, liposomes, microcapsules, microspheres, or oils.

For treatments of the eye or other external tissues, such as the mouth or skin, the formulations (e.g., a pharmaceutical composition comprising a Rho kinase inhibitor) may be applied as a topical ointment or cream. When formulated in an ointment, a Rho kinase inhibitor may be employed with either a paraffinic or a water-miscible ointment base.

Alternatively, a Rho kinase inhibitor may be formulated in a cream with an oil-in-water cream base or a water-in-oil base.

The present subject matter provides compositions comprising a Rho kinase inhibitor and a carrier or excipient suitable for administration to ocular tissue. Such carriers and excipients are suitable for administration to ocular tissue (e.g., sclera, lens, iris, cornea, uvea, retina, macula, or vitreous tissue) without undue adverse side effects (such as toxicity, irritation, and allergic response) commensurate with a reasonable benefit/risk ratio.

Pharmaceutical formulations adapted for topical administrations to the eye include eye drops wherein a Rho kinase inhibitor is dissolved or suspended in a suitable carrier, especially an aqueous solvent. Formulations to be administered to the eye will have ophthalmically compatible pH and osmolality. The term “ophthalmically acceptable vehicle” means a pharmaceutical composition having physical properties (e.g., pH and/or osmolality) that are physiologically compatible with ophthalmic tissues.

In some embodiments, the Rho kinase inhibitor is formulated as a nanoemulsion, e.g., as described in WO2019099595, e.g., for topical (eye drops) or injection administration. For example, a Rho kinase inhibitor can be encapsulated in nanoscale droplet/particles to form a nanoemulsion form of the inhibitor. In some embodiments, the particles comprise a polymer, for example, a biodegradable polymer, e.g., polycaprolactone (PCL). Additional biodegradable polymers widely used in the art include polyglycolic acid (PGA), polylactic acid (PLA), lactic acid-glycolic acid copolymer (PLGA), lactic acid-s-caprolactone copolymer (PLCL), polydioxanone (PDO), polytrimethylene carbonate (PTMC), poly(amino acid), polyanhydride, polyorthoester, polyvinyl alcohol, and copolymers thereof. In some embodiments, the particle comprises a length of 5-500 or 10-200 nanometers in at least one dimension.

In some embodiments, an ophthalmic composition of the present invention is formulated as sterile aqueous solutions having an osmolality of from about 200 to about 400 milliosmoles/kilogram water (“mOsm/kg”) and a physiologically compatible pH. The osmolality of the solutions may be adjusted by means of conventional agents, such as inorganic salts (e.g., NaCl), organic salts (e.g., sodium citrate), polyhydric alcohols (e.g., propylene glycol or sorbitol) or combinations thereof.

In some embodiments, the ophthalmic formulations of the present invention may be in the form of liquid, solid or semisolid dosage form. The ophthalmic formulations of the present invention may comprise, depending on the final dosage form, suitable ophthalmically acceptable excipients. In some embodiments, the ophthalmic formulations are formulated to maintain a physiologically tolerable pH range. In certain embodiments, the pH range of the ophthalmic formulation is in the range of from about 5 to about 9. In some embodiments, pH range of the ophthalmic formulation is in the range of from about 6 to about 8, or is about 6.5, about 7, or about 7.5.

In some embodiments, the composition is in the form of an aqueous solution, such as one that can be presented in the form of eye drops. By means of a suitable dispenser, a desired dosage of the active agent can be metered by administration of a known number of drops into the eye, such as by one, two, three, four, or five drops.

One or more ophthalmically acceptable pH adjusting agents and/or buffering agents can be included in a composition of the invention, including acids such as acetic, boric, citric, lactic, phosphoric, and hydrochloric acids; bases such as sodium hydroxide, sodium phosphate, sodium borate, sodium citrate, sodium acetate, and sodium lactate; and buffers such as citrate/dextrose, sodium bicarbonate, and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium, or ammonium cations and chloride, citrate, ascorbate, borate, phosphate, bicarbonate, sulfate, thiosulfate, or bisulfite anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g., poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition, and polymeric coatings that will enhance drug diffusion, erosion, dissolution, and osmosis.

Formulations for drug delivery using ocular devices may combine one or more active agents and adjuvants appropriate for the indicated route of administration. For example, a ROCK1/2 inhibitor (optionally with another agent) may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D,L-lactide-co-glycolide), poly (glycolide), poly (hydroxybutyrate), poly (alkylcarbonate) and poly (orthoesters), and mixtures thereof. Representative examples of non-biodegradable polymers can include EVA copolymers, silicone rubber and poly (methylacrylate), and mixtures thereof. See also Seah et al., Nat Biomed Eng. 2020 November; 4(11):1024-1025.

Pharmaceutical compositions for ocular delivery also include in situ gellable aqueous compositions. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig, Adv. Drug Deliv. Rev. 3; 57:1595-639 (2005), the entire content of which is incorporated herein by reference. Other ocular drug delivery systems can be used, e.g., as described in Robert et al., Transl Vis Sci Technol. 2016 March; 5(2): 11; Zhou et al., Invest Ophthalmol Vis Sci. 2017 January; 58(1): 96-105; Rawas-Qalaji and Williams, Curr Eye Res. 2012 May; 37(5):345-56.

Biocompatible implants for placement in the eye have been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; 6,699,493; and 8,293,210, the entire contents of each of which are incorporated herein by reference.

Drug-eluting contact lenses can also be used, e.g., as described in U.S. Pat. No. 8,414,912 and Ciolino et al., Invest Ophthalmol Vis Sci. 2009 July; 50(7): 3346-3352.

The implants may be monolithic, i.e. having the active agent (e.g., a ROCK1/2 inhibitor) or agents homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type implant may be of benefit in some circumstances, where the therapeutic level of the drug falls within a narrow window. In addition, the therapeutic component, including a ROCK1/2 inhibitor, may be distributed in a non-homogenous pattern in the matrix. For example, the implant may include a portion that has a greater concentration of a ROCK1/2 inhibitor relative to a second portion of the implant.

The intraocular implants disclosed herein may have a size of between about 5 um and about 2 mm, or between about 10 um and about 1 mm for administration with a needle, greater than 1 mm, or greater than 2 mm, such as 3 mm or up to 10 mm, for administration by surgical implantation. The vitreous chamber in humans is able to accommodate relatively large implants of varying geometries, having lengths of, for example, 1 to 10 mm. The implant may be a cylindrical pellet (e.g., rod) with dimensions of about 2 mm.times.0.75 mm diameter. The implant may be a cylindrical pellet with a length of about 7 mm to about 10 mm, and a diameter of about 0.75 mm to about 1.5 mm.

The implants may also be at least somewhat flexible so as to facilitate both insertion of the implant in the eye, such as in the vitreous, and accommodation of the implant. The total weight of the implant is usually about 250-5000 ug, more preferably about 500-1000 ug. For example, an implant may be about 500 ug, or about 1000 ug. For non-human subject, the dimensions and total weight of the implant(s) may be larger or smaller, depending on the type of subject. For example, humans have a vitreous volume of approximately 3.8 ml, compared with approximately 30 ml for horses, and approximately 60-100 ml for elephants. An implant sized for use in a human may be scaled up or down accordingly for other animals, for example, about 8 times larger for an implant for a horse, or about, for example, 26 times larger for an implant for an elephant.

Implants can be prepared where the center may be of one material and the surface may have one or more layers of the same or a different composition, where the layers may be cross-linked, or of a different molecular weight, different density or porosity, or the like. For example, where it is desirable to quickly release an initial bolus of drug, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The implants may be of any geometry including fibers, sheets, films, microspheres, spheres, circular discs, plaques, and the like. The upper limit for the implant size will be determined by factors such as toleration for the implant, size limitations on insertion, ease of handling, etc. Where sheets or films are employed, the sheets or films will be in the range of at least about 0.5 mm.times.0.5 mm, usually about 3-10 mm.times.5-10 mm with a thickness of about 0.1-1.0 mm for ease of handling. Where fibers are employed, the fiber diameter will generally be in the range of about 0.05 to 3 mm and the fiber length will generally be in the range of about 0.5-10 mm Spheres may be in the range of 0.5 um to 4 mm in diameter, with comparable volumes for other shaped particles.

The size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Microspheres for ocular delivery are described, for example, in U.S. Pat. Nos. 5,837,226; 5,731,005; 5,641,750; 7,354,574; and U.S. Pub. No. 2008-0131484, the entire contents of each of which are incorporated herein by reference.

For oral or enteral formulations for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. Nos. 4,704,295; 4, 556,552; 4,309,404; and 4,309,406, the entire contents of each of which are incorporated herein by reference.

In some embodiments, the ROCK1/2 inhibitor is formulated for sustained release. A number of sustained release formulations are known in the art, including but not limited to biodegradable implants such as lipid-encapsulated formulations, e.g., as described in Bonetti et al., Cancer Chemother Pharmacol 33:303-306 (1994) and Chatelut et al., J Pharm Sci. 1994 March; 83(3):429-32; multivesicular liposome (MVL) formulations, e.g., as described in WO2011143484; polymer microspheres, polymer-drug conjugates, or nano- or micropartricules, e.g., alpha-lactalbumin microparticles, e.g., as described in Vijayaragavan et al., Int J Pharm Res 3(1):39-44 (2011) or nanoparticles of drug conjugated with-human serum albumin as described in Taheri et al., J Nanomaterials 2011 (dx.doi.org/10.1155/2011/768201); polyion complex (PIC) micelles; bioadhesive polymers such as hydroxypropyl methylcellulose (HPMC), carboxymethylcellulose (CMC) and polyacrylic acid (PAA) derivatives, as well as hyaluronic acid (HA), e.g., Lacrisert (Aton Pharma), which is a soluble hydroxy propyl cellulose ocular insert.

Alternatively or in addition, sustained release can be achieved using a sustained-release device such as intravitreal implants, e.g., as described in Palakurthi et al., Current Eye Research, 35(12):1105-1115 (2010) or similar to the RETISERT (Bausch & Lomb), OZURDEX (Allergan); or non-biodegradable implants, e.g., similar to ILUVIEN (Alimera) or VITRASERT (Bausch & Lomb) implants, or the I-VATION platform (SurModics Inc.). See also Lee et al., Pharm Res. 27(10):2043-53 (2010); Haghjou et al., J Ophthalmic Vis Res. 6(4):317-329 (2011); Kim et al., Invest. Ophthalmol. Vis. Sci. 45(8):2722-2731 (2004); and Velez and Whitcup, Br J Ophthalmol 83:1225-1229 (1999). See also U.S. Pat. Nos. 4,704,295; 4, 556,552; 4,309,404; and 4,309,406.

See also US 20200377888; U.S. Pat. No. 10,828,306; PCT/US2017/061620; PCT/US2018/061110; PCT/US2018/061156; and PCT/US2015/042951, all of which are incorporated herein by reference in their entirety.

### Examples

The invention is further described in the following examples, which do not limit the scope of the invention described in the claims.

Materials and Methods

The following materials and methods were used in the Examples set forth herein.

Assessment of Proliferation and Cytotoxicity of Primary PVR Cultures

PVR cells were cultured and seeded into 96 well assay plates at 10,000 cells/well in complete growth media. Rho kinase inhibitors, Netarsudil, Ripasudil, Fasudil, Y27632 obtained from Selleck Chemicals (Houston, TX, USA) were dissolved in DMSO and used at two different concentrations. Cell proliferation was assessed at 24, and 48 hour time points by CyQUANT® Direct Cell Proliferation Assay (Thermofisher Scientific) and compared to vehicle controls at 24, and 48 hours post treatment. The data was expressed as percent cell inhibition.

Conditioned media was also collected at 24, and 48 hour time points for each condition, and percentage cell death was quantified by measuring LDH release using the CytoTox 96 Non-Radioactive Cytotoxicity Assay (Promega, Madison, WI, USA). As a positive control, the same number of cells maintained in parallel was lysed by two freeze—thaw cycles; the conditioned media were collected to measure the maximum LDH release. Percentage LDH release was calculated as 100%×(experimental LDH−spontaneous LDH)/(maximal LDH−spontaneous LDH).

Rho Kinase Inhibition in an Ex Vivo Model of PVR

PVR membranes were divided into pieces and embedded in growth factor reduced Matrigel (354,230; BD Biosciences, San Jose, CA) in a 24-well plate and placed at 37° C. for 30 min for the Matrigel to solidify. The specimens were treated with either vehicle or Rho kinase inhibitors, Netarsudil (60 nM), Ripasudil (300 nM), Fasudil (1 uM), and Y27632 (1 uM) in 500 μl in PVR growth media comprising:
1) Endothelial base medium (EBM2; Lonza-CC-3156)+
2) EGM 2 SingleQuot Kit suppl. & Growth factors (Lonza; CC-4176)+
3) 10% fetal bovine serum (Atlanta Biologicals)+
4) 1% Penicillin/Streptomycin+1% L-Glutamine
*** The total FBS in the media is 12% (2% from SingleQuot+10% additional)

Phase contrast images were taken using an EVOS FL imaging system (Life Technologies) and the distance of growth from the embedded tissue was quantified using Image J (National Institutes of Health).

Immunoprecipitation (IP) Protocol

C-PVR cells were cultured and seeded in T-25 flasks at 1 million cells per flask. Each flask was treated with Recombinant Human TGF-β2 (0.1 ug/ml) (PeproTech US) and collected at 0, 5, 30, and 60 minutes. Cells were washed with ice-cold PBS and cell lysates were harvested with a cell scraper. Protein concentration of each time point was measured to ensure equal protein concentration. Using freshly prepared cell lysates, equivalent protein amounts (300-500 ug total protein) were added to 30 ul of rhotekin-RBD beads (Cytoskeleton, Denver, CO) for the Rho-kinase pulldown assay. Pulldown assay supernatants were quantified using western blot analysis. An anti-RhoA primary antibody (Cytoskeleton, Denver, CO) was added and left overnight at 4° C., After primary antibody intubation, HRP labeled secondary antibody was added, following by addition on HRP detection reagent and intubated at room temperature for 5 minutes,

Western Blotting

Protein concentrations were measured, and equal concentrations of protein were separated using 4-20% SDS-PAGE (456-1094, Bio-Rad Laboratories, Hercules, CA), transferred to polyvinylidene difluoride membranes (Millipore Sigma, Darmstadt, Germany) and blocked using Odyssey Blocking Buffer (LI-COR Biosciences) for 1 h at room temperature. The membranes were incubated overnight at 4° C. with primary antibodies rabbit anti-Fibronectin (Sigma Aldrich) and mouse anti N-Cadherin (Santa Cruz Biotechnology). After washing, the membranes were probed with IRDye 680RD donkey anti-rabbit, and IRDye 800CW donkey anti-mouse (LI-COR Biosciences) antibodies for 1 h at room temperature. Immunoreactive bands were visualized using the Odyssey Infrared Imaging System, and band intensities normalized to rabbit anti-β-actin (Cell Signaling Technologies) were quantified using Image Studio (LI-COR Biosciences) using our previously developed protocol.

### Example 1. Effect of Rho-Kinase Inhibition on a Patient-Derived Model of Proliferative Vitreoretinopathy

To incorporate the complexity of cell types involved in the pathobiology of PVR, we decided to use primary cell cultures obtained from human PVR membranes. These primary cells, “PVR cells”, grow robustly in culture, retained the expression of cell identity markers in culture and form membranes and band-like structures in culture similar to the human condition. After a single cell RNA sequence analysis of our human collected PVR membranes, we found that ROCK1 and ROCK2 levels were upregulated in our sample. We decided to test the effects of Rho-kinase inhibition using FDA-approved ROCK1 and ROCK2 inhibitors: ripasudil, netarsudil, fasudil and Y-2762, and explore the role it played in the progression of PVR. Furthermore, we have previously shown the critical role epithelial to mesenchymal transition (EMT) has in the progression of PVR, showing that under a stimulus of growth factors, such as TGF-β2, EMT markers were upregulated. Rho-kinase activation has also been linked to play a role in the EMT of different diseases, but the link has never been made in EMT progression of PVR. In the Examples set forth herein, we explored the role of rho-kinase inhibition in progression of PVR.

PVR cells were seeded in well plates for a period of 72 hours. Cells were pre-treated with rho-kinase inhibitors: ripasudil, netarsudil, fasudil and Y-2762 (1 uM) for 24 hours, then treated with TGF-β2 (long/ml), rho-kinase inhibitors (1 uM), and control with no drug, using triplicates for each condition. Changes in phenotype were examined via phase images at day 3.

PVR cells were cultured using Rho-Kinase inhibitors ripasudil, netarsudil, fasudil, Y-27632 at different concentrations (0.1 μM, 10 μM) and control with no drug. Cell proliferation and cell death LDH levels were measured and quantified at 48 hours.

PVR cells cultured using Rho-Kinase inhibitors ripasudil (1 μM), netarsudil (1 μM), fasudil (1 μM), Y-27632 (1 μM) and control with no drug. Cell migration was measured and quantified with phase images at 0, 3, 6, and 12 hours.

PVR cells cultured in T-25 flasks and treated with TGF-β2 (long/ml) at 80% confluence. Lysates from cells were collected at different 0, 5, 30, and 60 minutes and RhoA activation was measured using a RhoA pull down activation kit (Cytoskeleton).

PVR membranes were obtained from human donors with PVR grade C undergoing surgery. Membrane fragments placed on a matrigel mount with PVR cell growth media and treated with Rho-Kinase inhibitors ripasudil (1 μM), netarsudil (1 μM), fasudil (1 μM), Y-27632 (1 μM) and control with no drug.

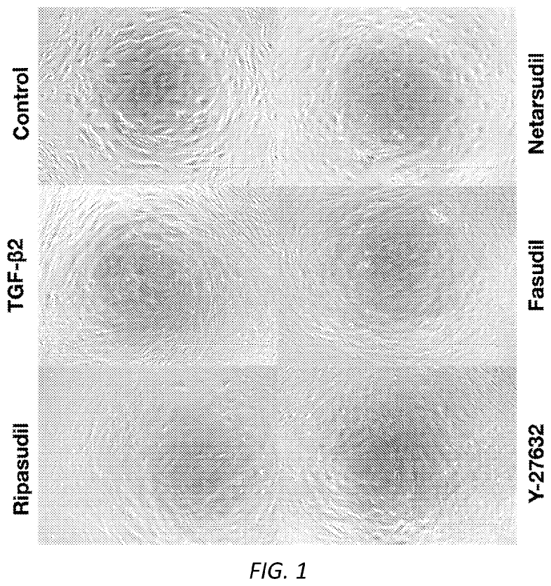

The effects of Rho kinase inhibitors Netarsudil, Ripasudil, Fasudil, Y27632 were evaluated in ex vivo cultured explants of PVR tissue from human subjects. As shown in FIG. 1, Fasudil and Y-27632 significantly reduced numbers of live cells in the explants.

### Example 1.1 Rho-Kinase Inhibition in TGF-β2 Treated C-PVR Cells Reduces Mesenchymal Phenotypic Change

TGF-β2 growth factor treatment (10 ng/ml) induced a mesenchymal phenotypic change in C-PVR cells, characterized by an elongated, fibroblast like phenotype. As shown in FIG. 1, treatment with Rho-Kinase inhibitors ripasudil, fasudil and Y-2762 (1 uM) slightly decreased the mesenchymal phenotypic change in C-PVR cells, while treatment with Rho-Kinase inhibitor netarsudil (1 uM) dramatically decreased mesechymal phenotypic change in C-PVR cells.

### Example 1.2. Rho-Kinase Inhibitors Ripasudil, Netarsudil, and Fasudil Decreased Proliferation in PVR Cells

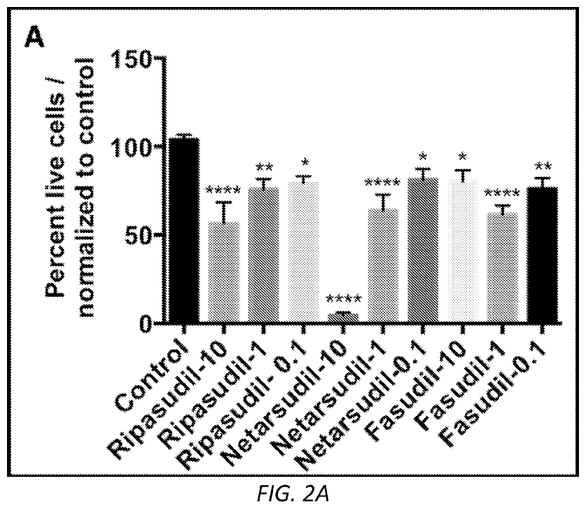

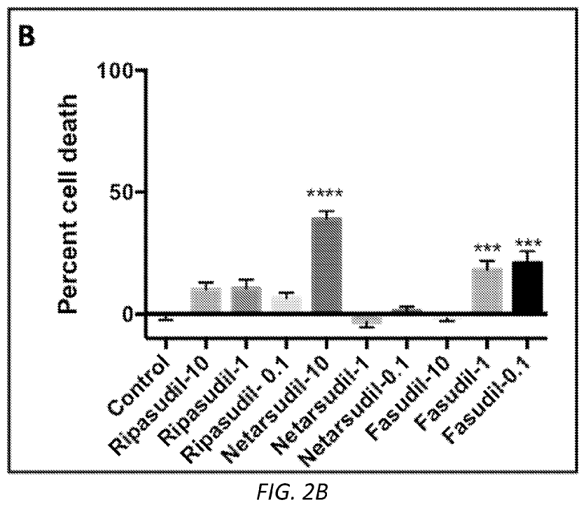

The ability of Rho-kinase inhibitors to decrease proliferation of PVR cells was evaluated. As shown in FIG. 2A, at 48 hours, significantly reduced proliferation in PVR cells by 44%, 95%, and 20% respectively at the highest concentration (10 μM), 25%, 37% and 39% percent reduction respectively with the lower concentration (1 μM), 21%, 39%, and 24% percent reduction respectively with the lowest concentration (0.1 μM). As shown in FIG. 2B, dose dependent cell death was detected by LDH analysis across concentrations with of ripasudil treatments and netarsudil treatments (10-0.1 μM), with low levels of cell death with fasudil treatment (10-0.1 μM),

### Example 1.3. Rho-Kinase Inhibitors Ripasudil, Netarsudil, and Fasudil Decrease Migration in PVR Cells

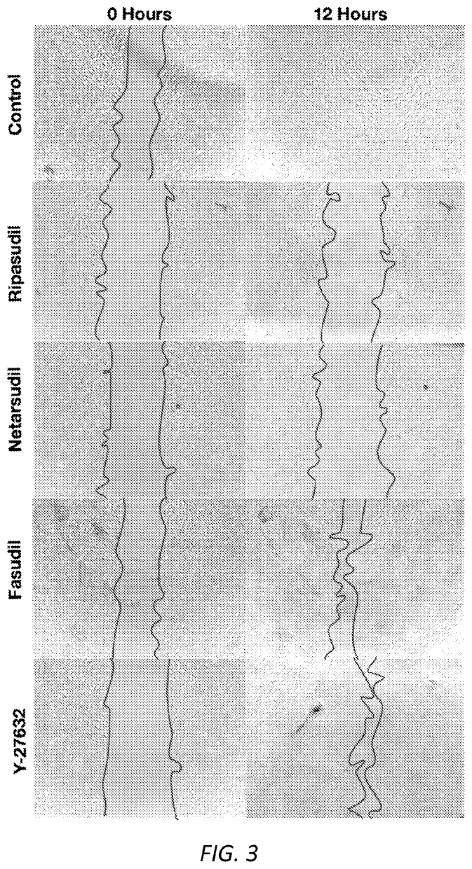

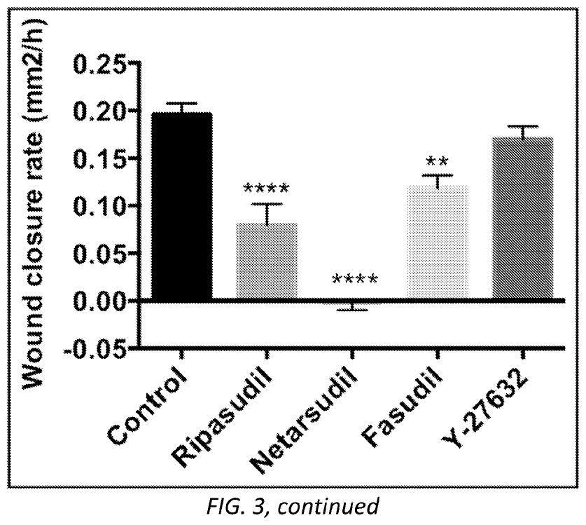

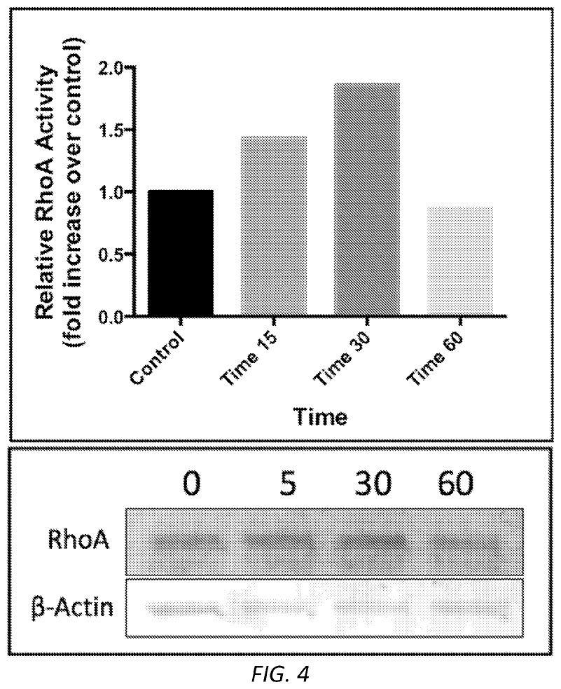

The ability of Rho-kinase inhibitors to decrease migration of PVR cells was evaluated. As shown in FIG. 3, significant cell migration was detected after 12 and 24 hours, but a decrease in cell migration between ripasudil, netarsudil, fasudil treatments and controls were detected. The graph shows that ripasudil, netarsudil and fasudil significantly reduced migration in C-PVR cells by 65%, 100%, and 40% respectively at a 1 μM concentration for all drugs.

### Example 1.4. TGF-β2 Induces RhoA Activation in PVR Cells, with Activation Peaking at 30 Minutes

This example evaluated effects of TGF-β2 treatment in C-PVR on RhoA activation. RhoA is known direct upstream activator of ROCK1 and ROCK2. TGF-β2 activation of RhoA had a 1.5 increase over control 15 minutes after stimulation, a 2 fold increase over control at 30 minutes, and a slight decrease over control at 60 minutes. These findings show that TGF-β2 RhoA activation peaks at 30 minutes and occurs in a dose-dependent fashion.

### Example 1.5. Rho-Kinase Inhibitors Decrease Proliferation and Migration of PVR Explants

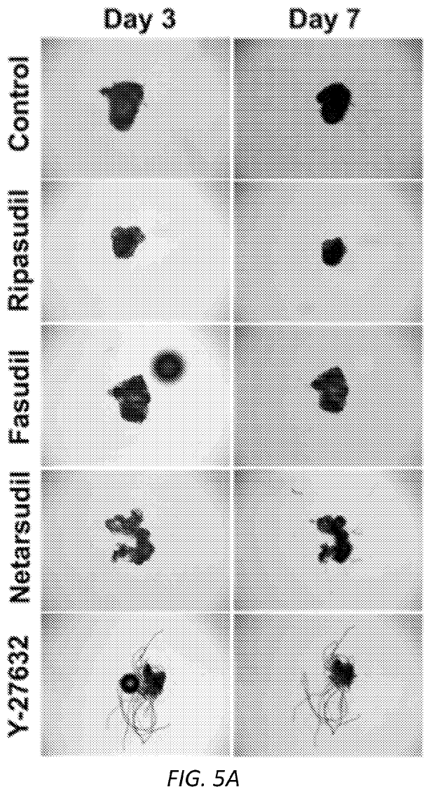

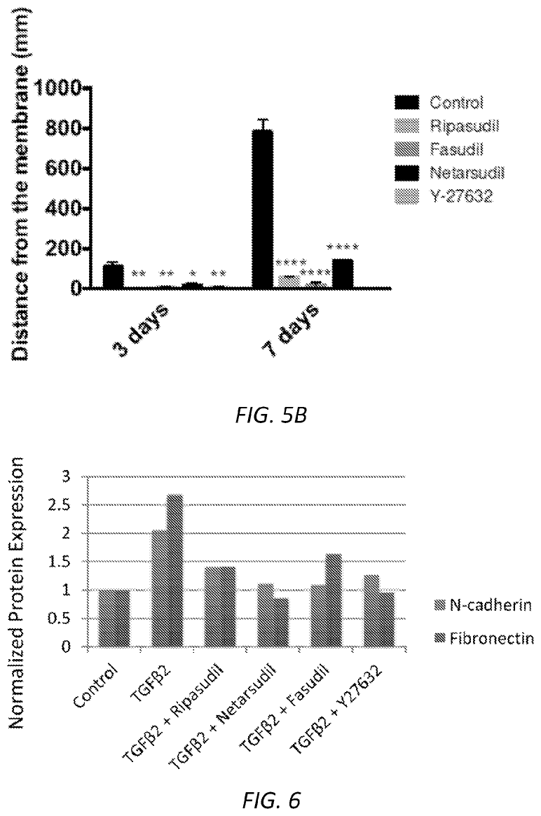

FIG. 5. Rho-Kinase inhibitors decrease proliferation and migration of PVR explants. PVR explants are patient-derived tissues placed into Matrigel after surgical removal from patients. Robust outgrowths were observed growing from the freshly isolated PVR explant samples at 7 and 14 days (28.58 mm and 207 mm respectively) post embedding in Matrigel in culture. ripasudil (0.8 mm and 15 mm), and netarsudil (4.2 mm and 37 mm) successfully inhibited and reduced explant growth at 7 and 14 days. The explants treated with fasudil (1 μM) and Y-2762 (1 μM) showed no outgrowths and almost complete inhibition of migration at all time points.

### Example 2. Rho Kinase Inhibition Decreased Expression of Markers of EMT

TGF-β has been shown to induce EMT (see, e.g., US 20200377888), and N-cadherin and fibronectin (markers of EMT) increase in the presence of TGF-β. As shown in FIG. 6, when evaluated by Western blot, treatment with ROCK inhibitors in PVR cells reduced the TGF-β-induced increases in N-cadherin and fibronectin.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or reducing the risk of proliferative vitreoretinopathy (PVR) or epiretinal membranes (ERM), or a condition associated with epithelial to mesenchymal transition (EMT), in a subject, the method comprising administering a therapeutically effective dose of a ROCK1/2 inhibitor.

2. The method of claim 1, comprising administering an intravitreal injection of a ROCK1/2 inhibitor.

3. The method of claim 1, wherein the ROCK1/2 inhibitor is administered posterior to the limbus.

4. The method of claim 1, wherein the subject is undergoing an ocular surgical procedure that increases the subject's risk of developing ERM or PVR.

5. The method of claim 4, wherein the ocular surgical procedure is a pars plana vitrectomy (PPV), Retinal Detachment (RD) surgery; ERM surgery; scleral buckle surgery; or a procedure in the other eye.

6. The method of claim 5, wherein the subject requires a PPV to treat a primary rhegmatogenous retinal detachment; rhegmatogenous retinal detachment secondary to trauma; preexisting proliferative vitreoretinopathy; or has other indications associated with high risk condition for PVR development.

7. The method of claim 6, wherein the indication associated with high risk condition for PVR development is a giant retinal tear, a retinal break larger than 3 disc areas, a long-standing retinal detachment, or a detachment associated with hemorrhage.

8. The method of claim 5, wherein:
a first injection is given at conclusion of the surgical procedure; and
at least one, two, three, four, or more weekly injections are given postoperatively.

9. The method of claim 1, comprising intravitreally administering a sustained release formulation of ROCK1/2 inhibitor.

10. The method of claim 9, wherein the sustained release formulation is or comprises a lipid-encapsulated formulation; multivesicular liposome (MVL) formulations; nano- or microparticles; polyion complex (PIC) micelles; or bioadhesive polymers.

11. The method of claim 10, wherein the bioadhesive polymers comprise one or more of hydroxypropyl methylcellulose (HPMC), carboxymethylcellulose (CMC), polyacrylic acid (PAA), or hyaluronic acid (HA).

12. The method of claim 1, wherein the inhibitor reduces the extent of or reverses proliferative vitreoretinopathy (PVR) or epiretinal membranes (ERM).

13. The method of claim 1, wherein the condition associated with EMT is cancer, ocular chronic graft-versus-host disease, corneal scarring, corneal epithelial downgrowth, conjunctival scarring, eye tumors like melanoma, ocular fibrosis, fibrosis, and complication of glaucoma surgery and/or aberrant post-surgical fibrosis, glaucoma, conjunctival fibrosis, or orbital fibrosis as found in thyroid eye disease.

14.-21. (canceled)
